(19) United States
(12) Patent Application Publication
(10) Pub. No.: US 2014/0323853 A1
(43) Pub. Date: Oct. 30, 2014

(54) MICROSTRUCTURE FOR ACOUSTIC DETECTION

(71) Applicant: NATIONAL TAIWAN UNIVERSITY, Taipei (TW)

(72) Inventors: Pai-Chi LI, Taipei (TW); I-Chin WU, Taipei (TW)

(73) Assignee: NATIONAL TAIWAN UNIVERSITY, Taipei (TW)

(21) Appl. No.: 13/966,576

(22) Filed: Aug. 14, 2013

(30) Foreign Application Priority Data
Apr. 26, 2013 (TW) 102115021

(51) Int. Cl. A61B 8/08

(52) U.S. Cl. CPC A61B 8/0841
USPC 600/424

(57) ABSTRACT

A microstructure applied to an invasive device which is set in an organism. The microstructure comprises at least two steps which is used to reflect an ultrasound signal to generate an echo signal to produce a location result according to the echo signal as an ultrasound probe transmits the ultrasound signal to the organism wherein the echo signal includes a wave that specific spectral characteristics can be achieved and utilized for effective detection.

### FIELD OF THE INVENTION

The present invention relates to a microstructure for acoustic detection, and more particularly relates to a microstructure for acoustic detection for generating an echo signal having a spectrum with a location feature.

### BACKGROUND OF THE INVENTION

Attending with the technological development and progress, acoustic detection technology, such as ultrasonic imaging technology, has been widely used in the modern diagnostic procedure. In compared with other medical imaging systems being used in clinical medicine, such as X-ray, CT, MRI or nuclear medicine imaging, the ultrasonic imaging technology has the advantages of low price, non-invasive, no danger of radiation, real-time imaging, mm level spatial resolution, portability, and blood detectable, and thus is widely used in clinical diagnosis of various departments.

Take amniocentesis for example, when doing the invasive procedure, the position of the needle should be detected in real time to make sure the other tissues and the baby would not be hurt. Although ultrasonic imaging can be used to assist the procedure of amniocentesis, in practice, the operation of the needle is still relied on the skill and experience of the operator. As a result, amniocentesis still carries some risk regarding damaging the other tissues and needs further improvement.

In addition, take the implants for example, an implantable device is used to detect the physiological signal in the body or assist the function of human organs, such as the middle-ear implant, into the body. The implant can be charged or transmit data by using the ultrasonic wave. Thus, precise location for the implant will significantly influence the correctness and effectiveness of the ultrasonic signal. Traditionally, an outside ultrasonic transceiver is used to detect the mechanical power to which the electric power is transferred by using the implant, so as to locate the implant.

Several detection methods have been proposed but all have some limitations in clinical applications due to the lack of precision or the need for power consumption, however, take the implant for example, a smaller rechargeable implant is better for reducing risk but will limit electric power capacity. Once the electricity runs out, the implant would not be detectable for executing the following charging or data transmission operations and thus result in the inconvenience of usage.

### BRIEF SUMMARY OF INVENTION

Because the traditional positioning technology for the invasive medicine device is not ideal, there exists some problems such as the additional risk of amniocentesis to damage the other tissues and the power exhausting problem of the implant. Accordingly, it is a main object of the present invention to provide a microstructure for reflecting the ultrasonic wave and having the reflected echo signal showing a spectrum with a feature to determine the position of the invasive device so as to enhance the correctness and effectiveness of the positioning technology.

A microstructure for acoustic detection is applicable for penetrating into a body and being positioned to generate a location result. The microstructure comprises at least two levels. When an ultrasonic signal is emitted by an ultrasonic probe toward the body, the levels are utilized for reflecting the ultrasonic signal to generate an echo signal having a spectrum with a location feature for generating the location result.

According to an embodiment of the present invention, the microstructure comprises two levels with a level difference therebetween, and the level difference causes destructive interference to the echo signal under a predetermined frequency in the spectrum. The location result is generated through accessing a difference value between the echo signal under the predetermined frequency and the echo signal under a frequency different from the predetermined frequency and the invasive device is positioned as the difference value is greater than a predetermined threshold value.

In accordance with an embodiment of the present invention, the microstructure comprises more than two levels, and the spectrum generated by using time-frequency analysis has the location feature showing a characteristic curve with a reducing frequency with time. In addition, each level of the microstructure has a level width x and a level height y, the ultrasonic probe is away from the microstructure with a minimum distance d, and a wavelength of the center frequency of an ultrasonic transducer is h. As the ultrasonic probe is a non-focusing probe, a total transmission distance S of the ultrasonic signal and the echo signal with respective to the ith level of the microstructure can be obtained by using Pythagorean theorem and satisfying the function S(i)=2{square root over ((d+(i−1)*x)²+((i−1)*y)²)}, and the wave length h is substantially identical to a difference ΔS of total transmission distance with respective to two neighboring levels.

Moreover, in accordance with an embodiment of the present invention, after the echo signal is received by an ultrasonic receiver, a depth range in the body is selected and a starting point and an end point of the echo signal is determined, then the echo signal is analyzed by using the time-frequency analysis to extract the characteristic curve for comparing with a simulated result stored in a database to generate a first correlation coefficient, and the location result is generated when the first correlation coefficient is greater than a threshold value. In addition, when deter mining the starting point and the end point of the echo signal, a time length after the starting point is further determined, and then the echo signal within the time length is analyzed by using the time-frequency analysis to generate the characteristic curve for comparing with the simulated result stored in the database to generate a second correlation coefficient, and the location result is generated when the second correlation coefficient is greater than the threshold value.

In accordance with an embodiment of the present invention, the threshold value is ranged from 0.5 to 1, and the time-frequency analysis is carried out by using an transformation selected from a group including short-time Fourier transform, wavelet transform, and Hilbert-Huang transform.

Thus, with the microstructure on the invasive device such as the needle or the implant, the position of the invasive device can be determined when the echo has the characteristic that the frequency decreases with time. In addition, the implant does not need power consumption for detection such that the problem of running out of power can be resolved and the size can be further reduced to solve the problem of the prior art.

The embodiments adopted in the present invention would be further discussed by using the flowing paragraph and the figures for a better understanding.

### BRIEF DESCRIPTION OF THE DRAWINGS

FIG. 1 is a schematic view of an invasive structure with a microstructure in accordance with a preferred embodiment of the present invention.

FIG. 2 is a flowchart showing the generation of the location result according to the echo signal in accordance with a preferred embodiment of the present invention;

FIG. 3 is a diagram showing a waveform of an simulated ultrasonic signal in accordance with a preferred embodiment of the present invention;

FIG. 3A is a diagram showing a simulated echo signal in accordance with a preferred embodiment of the present invention;

FIG. 3B is a diagram showing the simulated result of the simulated echo signal after time-frequency analysis in accordance with a preferred embodiment of the present invention;

FIG. 4 is a diagram showing a waveform of an experimental echo signal in accordance with a preferred embodiment of the present invention;

FIG. 4A is a diagram showing the experimental characteristic curve of the experimental echo signal after time-frequency analysis in accordance with a preferred embodiment of the present invention;

FIG. 4B is a schematic view showing a comparison of the simulated result and the experimental characteristic curve in accordance with a preferred embodiment of the present invention;

FIG. 5 is a schematic view showing an invasive device with a microstructure applied to a needle in accordance with a first embodiment of the present invention;

FIG. 6 is a schematic view showing an invasive device with a two-stepped microstructure applied to a needle in accordance with a second embodiment of the present invention;

FIG. 6A is a cross-section view showing the invasive device with a two-stepped microstructure applied to a needle in accordance with the second embodiment of the present invention; and

FIG. 7 is a schematic view showing the microstructure applied to an implant in accordance with a preferred embodiment of the present invention.

### DETAILED DESCRIPTION OF THE INVENTION

There are various embodiments of the invasive device with the microstructure in accordance with the present invention, which are not repeated hereby. The preferred embodiments are mentioned in the following paragraph as an example. It should be understood by those skilled in the art that the preferred embodiments disclosed in the following paragraph are merely an example instead of restricting the scope of the invention itself.

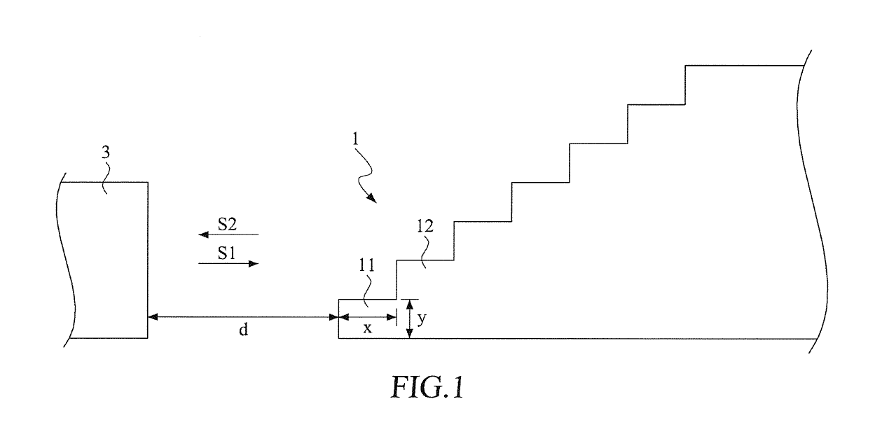

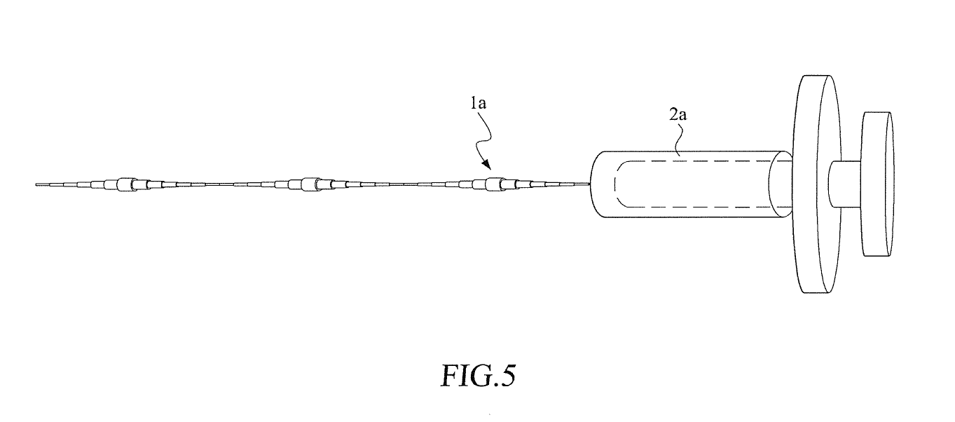

FIG. 1 shows a schematic view of an invasive structure with a microstructure in accordance with a preferred embodiment of the present invention. As shown, the invasive device 2a, 2b, 2c (please refer to FIGS. 5, 6, and 7) utilized to be positioned in a body (not shown) has a microstructure 1 for positioning the invasive device 2a, 2b, 2c to generate a location result. In the present embodiment, the body can be a human body, and the invasive device 2a, 2b, 2c can be a needle or an implant. However, the present invention is not so restricted.

In the present embodiment, the microstructure 1 includes seven levels. Take the first level 11 and the second level 12 for example to better describe the present embodiment, the first level 11 and the second level 12 has the identical level width x and the identical level height y. In addition, the ultrasonic probe 3 is away from the first level 11 with a minimum distance d and the ultrasonic probe 3 is a non-focusing probe, which emits an ultrasonic signal S1 with a center frequency wavelength h. It should be noted that, in the present embodiment, the minimum distance d means a smaller linear distance from the first level 11 to the ultrasonic probe 3 and the center frequency wavelength h substantially equals to a difference ΔS of total transmission distance of the ultrasonic signal and the echo signal with respective to two neighboring levels.

The first level 11 and the second level 12 are utilized for reflecting the ultrasonic signal S1, which is generated by the ultrasonic probe 3 toward the body, to generate an echo signal S2 for generating the location result accordingly. The echo signal shows a characteristic curve with a reducing frequency attending with an increasing time under time-frequency analysis.

Basically, since the horizontal and the vertical distance is known, the total transmission distance S can be calculated by using Pythagorean theorem. In detail, the total transmission distance S of the ultrasonic signal Si and the echo signal S2 of the ith level of the seven levels satisfies the function: S(i)=2{square root over ((d+(i−1)*x)²+((i−1)*y)²)}. Take the first level 11 for example, the total transmission distance S equals to the moving distance of the ultrasonic signal S1 plus the moving distance of the echo signal S2, the total transmission distance S with respective to the first level 11 is S(1)=2{square root over ((d+(1−1)*x)²+((1−1)*y)²)}, and thus S(1)=2\sqrt{(d)²}=2d. Similarly, the total transmission distance with respective to the second level 12 can be calculated through replacing i with 2 into the above mentioned functions.

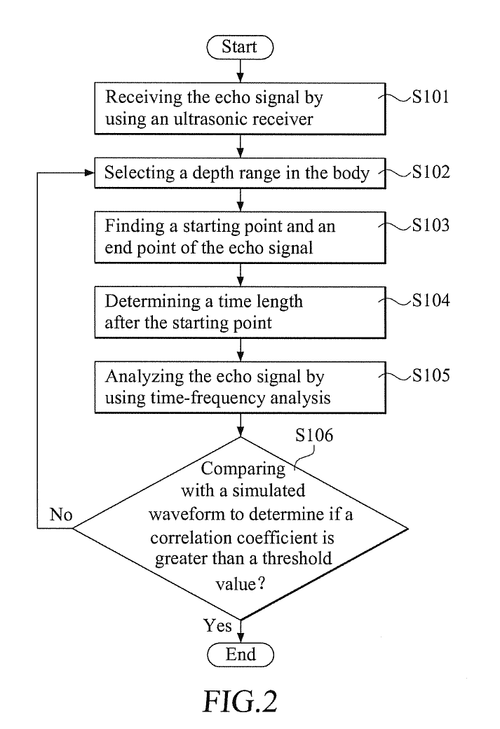

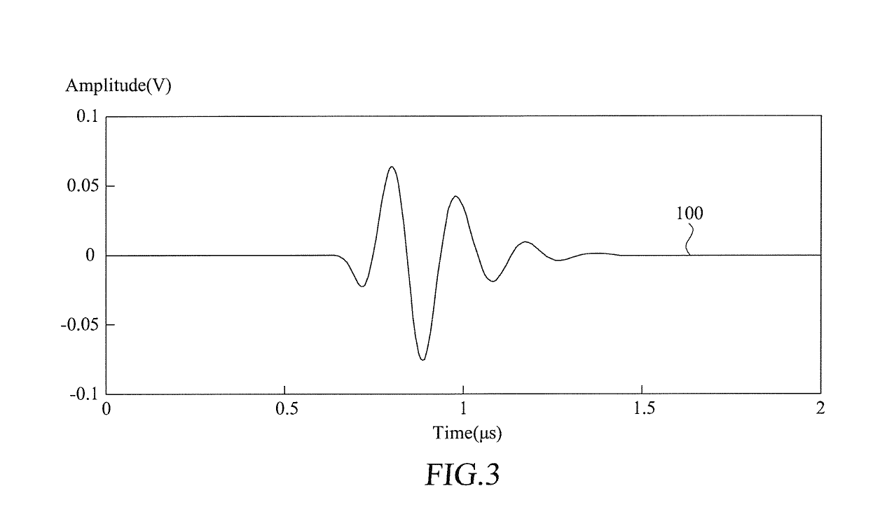

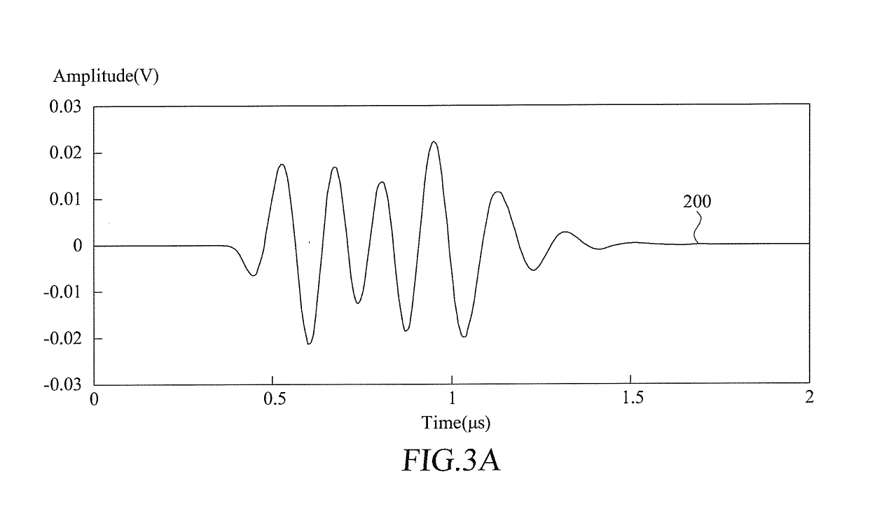

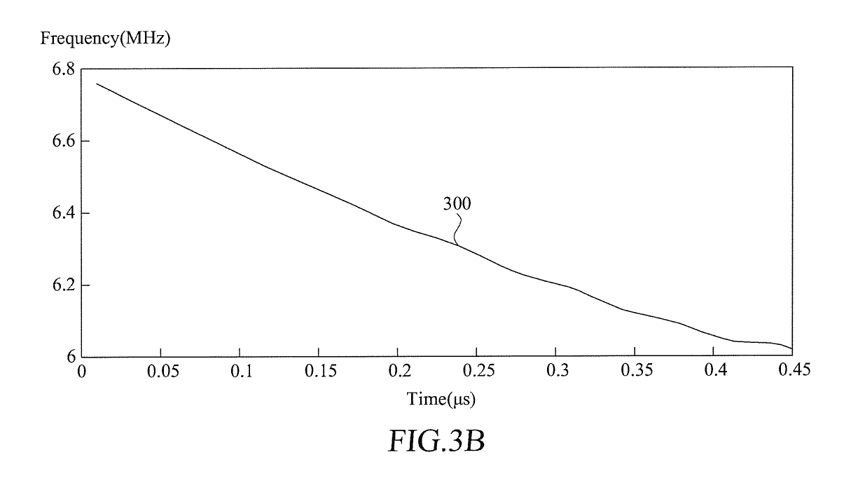

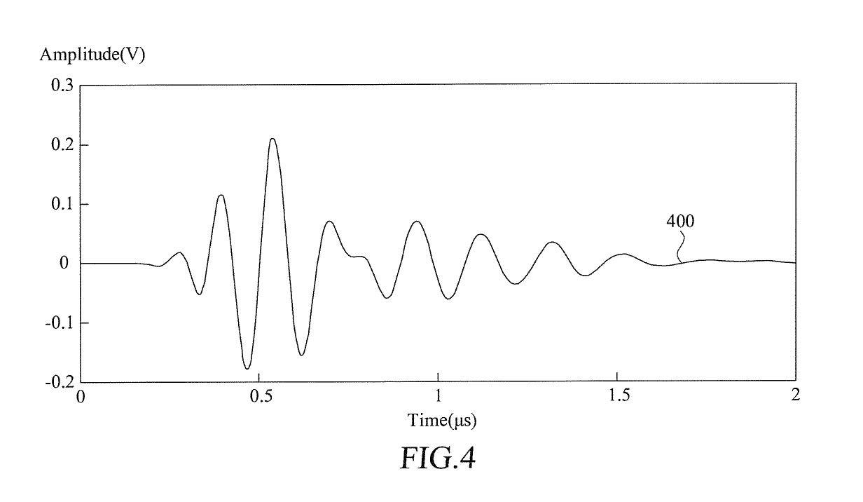

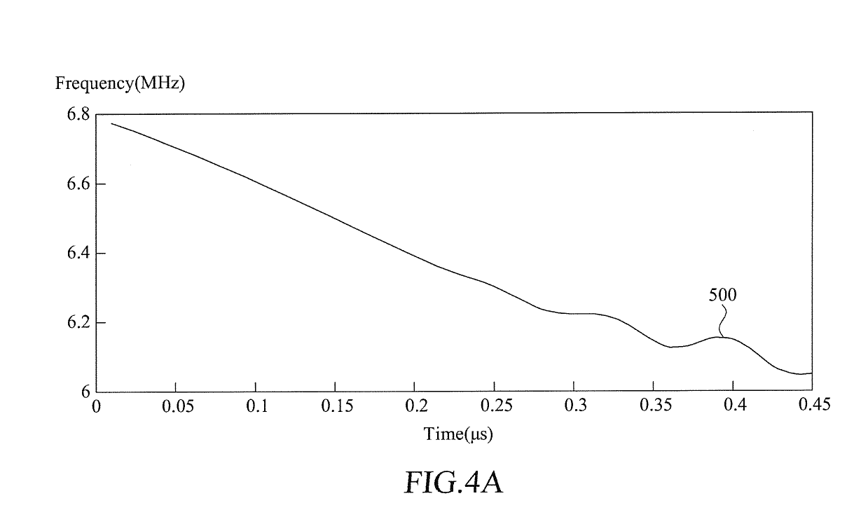

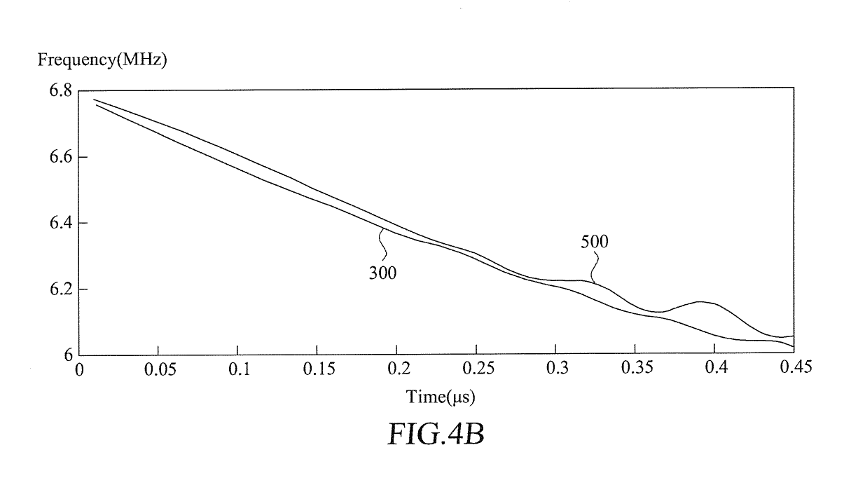

Please refer to FIGS. 1 to 4B for a better understanding of the present invention, wherein FIG. 2 is a flowchart showing the generation of the location result according to the echo signal in accordance with a preferred embodiment of the present invention, FIG. 3 is a diagram showing a waveform of an simulated ultrasonic signal in accordance with a preferred embodiment of the present invention, FIG. 3A is a diagram showing a simulated echo signal in accordance with a preferred embodiment of the present invention, FIG. 3B is a diagram showing the simulated result of the simulated echo signal after time-frequency analysis in accordance with a preferred embodiment of the present invention, FIG. 4 is a diagram showing a waveform of an experimental echo signal in accordance with a preferred embodiment of the present invention, FIG. 4A is a diagram showing the experimental waveform of the experimental echo signal after time-frequency analysis in accordance with a preferred embodiment of the present invention, and FIG. 4B is a schematic view showing a comparison of the simulated result and the experimental waveform in accordance with a preferred embodiment of the present invention. The location result is generated according to the echo signal by using the process including the steps of:

Step S101: receiving the echo signal by using an ultrasonic receiver;

Step 102: selecting a depth range in the body

Step 103: finding a starting point and an end point of the echo signal;

Step 104: determining a time length after the starting point.

Step 105: analyzing the echo signal by using the time-frequency analysis to generate a characteristic curve; and

Step S106: comparing with a simulated result to determine if a correlation coefficient is greater than a threshold value.

After the process begins, the echo signal S2 is received by an ultrasonic receiver (not shown) in step S101, and then a depth range is selected in the body in step S102. The depth range may be a few centimeters into the body for example, however, the present invention is not so restricted. The ultrasonic receiver can be any receiver capable of receiving the ultrasonic echo signal, which should be well understood for the person skilled in the art and thus is skipped here.

After the step S102 is finished, step S103 is carried out to determine a starting point and an end point of the echo signal S2. In detail, this step is to locate a section of the waveform of the echo signal S2 from the starting point, where the amplitude appears, to the end point, where the amplitude disappears. After locating the starting point, the step S104 is carried out to access the waveform of the echo signal S2 within a time length, such as 0.45 μs, after the starting point. However, the present invention is not so restricted.

After accessing the section of the waveform, the step S105 is executed to analyze the echo signal S2 by using time-frequency analysis, especially the echo signal S2 within the above mentioned time length. The time-frequency analysis is carried out by using the transformation selected from a group including short-time Fourier transform, wavelet transform, and Hilbert-Huang transform so as generate a characteristic curve showing a reducing frequency attending with an increasing time. However, the present invention is not so restricted.

After the step S105, the step S106 is carried out to compare the characteristic curve generated in step S105 with a simulated result stored in a database so as to generate a correlation coefficient and judge if the correlation coefficient is greater than a threshold value. The database can be a memory or other hardware with storing ability. The correlation coefficient represents the correlation between the characteristic curve generated in step S105 and the simulated result. Thus, the correlation coefficient can be used to determine if the characteristic curve is close to the simulated result, and the threshold value can be set to optimize the result. In the present embodiment, the threshold value is ranged from 0.5 to 1, and in practice, the threshold value can be set as 0.9. If the judging result in step 106 is yes, the correlation coefficient is greater than the threshold value, which implies that the invasive device 2a, 2b, 2c should be located within the depth range selected in step S101 so as to generate the location result.

If the judging result in step S106 is no, there should be no invasive device 2a, 2b, 2c in the depth range. Then the steps S102 to S105 are repeated. In addition, the step S104 may be skipped and the step S105 can be executed directly. The purpose of step S104 is to select the data within a certain time length for further analysis in step S106. Thus, this should be an optional step according to the need in practice.

Moreover, the simulated result stored in the database as described in step S106 can be a predefined waveform, such as the simulated result data generated through running the simulation many times. Concretely speaking, as shown in FIG. 3, as a preferred embodiment of the present invention, the aperture of the ultrasonic probe 3 is set to be 0.5 inch. When running the simulation, the ultrasonic probe 3 emits a simulated ultrasonic signal for simulating the waveform 100 of the ultrasonic signal to the microstructure 1 of the invasive device, and the simulated ultrasonic signal is reflected to generate the simulated echo signal for simulating the waveform 200 of the echo signal, and after the process as shown in FIG. 2, a simulated result 300 showing a feature of a reducing frequency attending with an increasing time is generated for storing in the database as mentioned in step S106.

FIG. 4A shows the first experimental characteristic curve 500 generated through analyzing the experimental echo signal with a waveform 400 similar to that shown in FIG. 4 by using time-frequency analysis. The experimental echo signal is generated by actually running an experiment. As shown in FIG. 4A, the experimental characteristic curve 500 shows the feature of a reducing frequency attending with an increasing time, which is similar to the simulated result 300. The experimental characteristic curve 500 as shown in FIG. 4B can be generated, which is quite close to the simulated result 300 for determining the existence of invasive device 2a, 2b, 2c within the selected depth range. Therefore, the effectiveness and industrial value of the present invention can be approved. The following paragraphs describes the needle and the implant applied to amniocentesis as a example to show the application of the present invention.

FIG. 5 is a schematic view showing an invasive device with a microstructure applied to a needle in accordance with a first embodiment of the present invention. As shown in FIG. 5, as the invasive device 2a is an amniocentesis needle, the microstructure 1a of the present invention can be generated through changing the outer radius of the needle. In the present embodiment, the needle shows a multi-level microstructure for generating an echo signal featuring the phenomenon that the frequency is reduced with time in response to the ultrasonic signal emitted from the outside such that the position of the needle can be determined. Therefore, the invasive devices without the capability to vibrate or emit signals, such as the needle, can be detected by the outside ultrasonic transceiver.

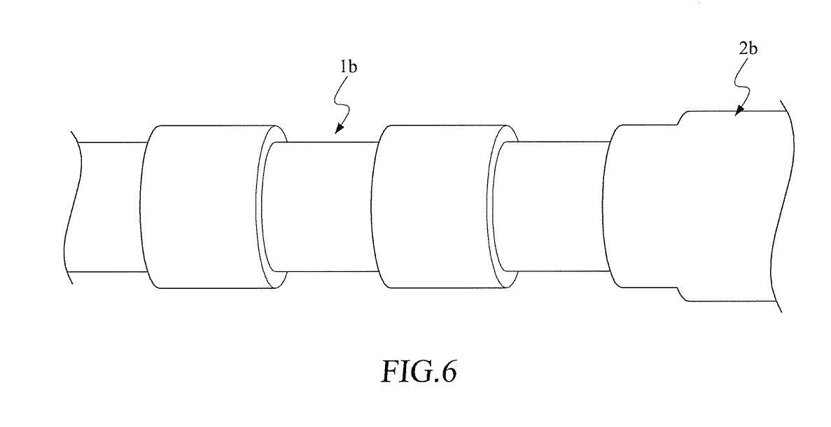

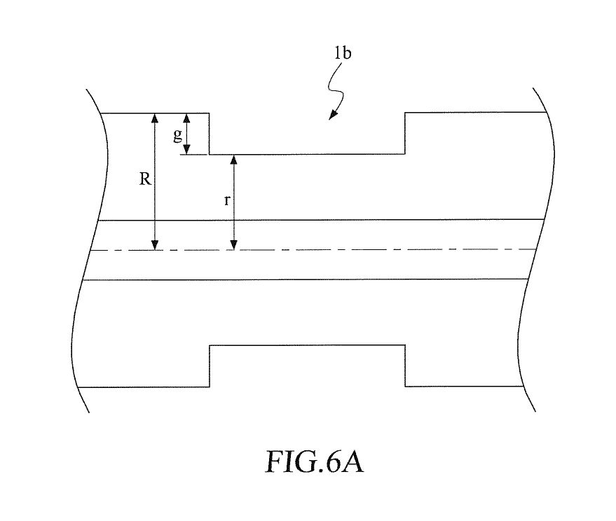

FIG. 6 is a schematic view showing an invasive device with a two-stepped microstructure applied to a needle in accordance with a second embodiment of the present invention. As shown in FIG. 6, the invasive device 2b is also an amniocentesis needle as shown in FIG. 5, but only has two different outer radiuses to form a two-level microstructure 1b. Thus, the position of the needle can be determined by using the analysis of constructive interference or destructive interference under certain frequency.

In detail, since there exists a level difference between the two levels, which causes destructive interference to the echo signal under a predetermined frequency in the spectrum. The location result can be generated through judging if a difference value between the echo signal under the predetermined frequency and the echo signal under a frequency different from the predetermined frequency is greater than a predetermined threshold value so as to determine the position of the invasive device.

FIG. 6A is a cross-section view showing the invasive device with a microstructure applied to a needle in accordance with the second embodiment of the present invention. As shown, there exists a level difference g between the level with a greater radius R and the level with a smaller radius r. In the present embodiment the level different g is identical to a quarter of a predetermined wave length. As the ultrasonic signal (not shown) propagated to the invasive device 2b, the echo signal (not shown) generated by the two-stepped microstructure will have destructive interference under the frequency with respective to the predetermined wave length and will have constructive interference with respective to double or half the frequency. Thus, after creating packets to include the echo signals of two different frequencies and subtracting the data of the packets, if the difference value is greater than the predetermined threshold value, it can be determined that the invasive device 2b is positioned right in front of the probe.

In detail, take the sonic speed of soft tissue 1540 m/s for example, the destructive interference happens at the frequency of 5 MHz, and the level difference g is set to be a quarter of the wavelength with respective to the frequency of 5 MHz, which is 77 μm, for causing the destructive interference. Thus, to packet and subtract the echo signals of this frequency and the other frequency, a difference value can be generated. As the difference value is greater than the threshold value, the invasive device should be positioned in front of the probe, and the time interval from emitting the ultrasonic signal till receiving the echo signal can be used to determined the depth so as to determined the position of the invasive device.

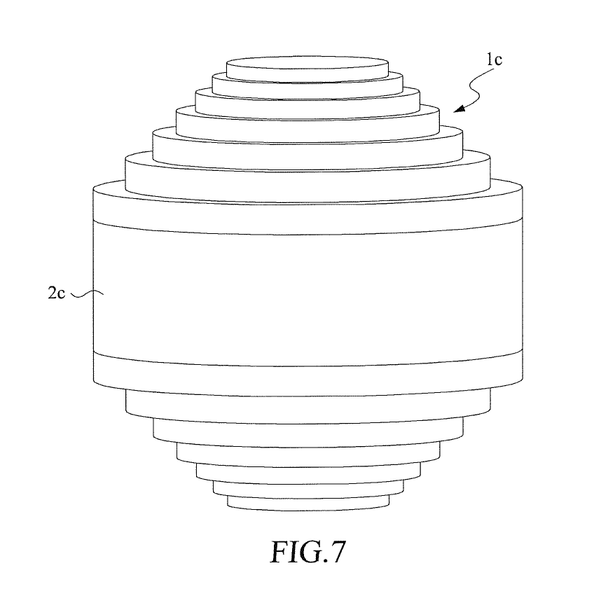

FIG. 7 is a schematic view showing the microstructure applied to an implant in accordance with a preferred embodiment of the present invention. As shown in FIG. 7, the invasive device 2c is an implant. After implanting the implant into the body, an echo signal featuring the phenomenon that the frequency is reduced with time would be generated in response to the ultrasonic signal emitted from the outside such that the position of the implant can be determined. In the present embodiment, the implant has the microstructures 1c formed on two opposite sides thereof. However, the present invention is not so restricted. The microstructure may be formed on one side or more than two sides of the implant. The greatest advantage of applying the microstructure to the implant is that the implant can be detected and located from the outside merely through the surface structure and no electric power is consumed. On the other hand, for the those can be charged by the ultrasonic wave, the present invention is helpful for detection the implants before the charging process.

In conclusion, with the microstructure on the invasive device such as the needle or the implant, the position of the invasive device can be determined after the characteristic curve featuring a reducing frequency with time is detected. In addition, the implant can be detected without any power consumption, and the stable wireless transmission can be performed.

The detail description of the aforementioned preferred embodiments is for clarifying the feature and the spirit of the present invention. The present invention should not be limited by any of the exemplary embodiments described herein, but should be defined only in accordance with the following claims and their equivalents. Specifically, those skilled in the art should appreciate that they can readily use the disclosed conception and specific embodiments as a basis for designing or modifying other structures for carrying out the same purposes of the present invention without departing from the scope of the invention as defined by the appended claims.

What is claimed is:

1. A microstructure for acoustic detection, applicable for penetrating into a body and being positioned to generate a location result, comprising: at least two levels, when an ultrasonic signal is emitted by an ultrasonic probe toward the body, utilized for reflecting the ultrasonic signal to generate an echo signal having a spectrum with a location feature for generating the location result.

2. The microstructure of claim 1, wherein the two levels have a level difference therebetween, and the level difference causes destructive or constructive interference to the echo signal under a predetermined frequency in the spectrum.

3. The microstructure of claim 2, wherein the location result is generated through receiving a difference value from the echo signal under the predetermined frequency and the echo signal under a frequency different from the predetermined frequency, and the invasive device is positioned as the difference value is greater than a predetermined threshold value.

4. The microstructure of claim 1, wherein the microstructure comprises more than two levels, and the spectrum generated by using time-frequency analysis has the location feature showing a characteristic curve with a reducing frequency attending with an increasing time.

5. The microstructure of claim 4, wherein each level of the microstructure has a level width and a level height, and the ultrasonic probe is away from the microstructure with a minimum distance such that a total transmission distance of the ultrasonic signal and the echo signal with respective to a certain level of the microstructure is obtained thereby.

6. The microstructure of claim 5, wherein a wavelength h of an ultrasonic transducer is substantially equal to a difference of total transmission distance with respective to two neighboring levels.

7. The microstructure of claim 5, wherein after the echo signal is received by an ultrasonic receiver, a depth range in the body is selected and a starting point and an end point of the echo signal is located, then the echo signal is analyzed by using the time-frequency analysis to generate the characteristic curve for comparing with a simulated result stored in a database to generate a first correlation coefficient, and the location result is generated when the first correlation coefficient is greater than a threshold value.

8. The microstructure of claim 7, wherein a time length after the starting point is determined when locating the starting point and the end point, then the echo signal is analyzed by using the time-frequency analysis to extract the characteristic curve for comparing with the simulated result stored in the database to generate a second correlation coefficient, and the location result is generated when the second correlation coefficient is greater than the threshold value.

9. The microstructure of claim 7, wherein the time-frequency analysis is carried out by using an transformation selected from a group including short-time Fourier transform, wavelet transform, and Hilbert-Huang transform.

10. The microstructure of claim 7, wherein the threshold value is ranged between 0.5 to 1.
